(19) United States
(12) Patent Application Publication
(10) Pub. No.: US 2017/0251889 A1
(43) Pub. Date: Sep. 7, 2017

(54) PRODUCT CONTAINER HOLDER

(71) Applicants: Nancy Carol Gawlik, Janesville, WI (US); Richard Edmund Koval, Elkhorn, WI (US)

(72) Inventors: Nancy Carol Gawlik, Janesville, WI (US); Richard Edmund Koval, Elkhorn, WI (US)

(21) Appl. No.: 15/451,932

(22) Filed: Mar. 7, 2017

Related U.S. Application Data
(63) Continuation-in-part of application No. 29/593,022, filed on Feb. 5, 2017.
(60) Provisional application No. 62/389,701, filed on Mar. 7, 2016.

(51) Int. Cl. A47K 10/18; A47K 5/12; A61B 42/40; B65D 25/04

(52) U.S. Cl. CPC A47K 10/185; B65D 25/04; A47K 5/12; A61B 42/40

(57) ABSTRACT

The present invention relates in general to the field of devices configured to hold containers used to store product to be distributed as well as the ornamental appearance of such devices. The invention comprises a wall mounted structure comprising at least one wall and one support surface where the support surface defines a void configured for allow a product stored in a box supported by the supported surface to extend through the void for ease of access.

### CLAIM TO PRIORITY

This application claims priority to provisional application 62/389,701, filed on 7 Mar. 2016, and is a continuation in part to application Ser. No. 29/593,022, filed on 5 Feb. 2017, of which the entire contents of such documents are hereby incorporated by this reference for all that the documents disclose for all purposes.

### TECHNICAL FIELD

Embodiments of the present invention relate in general to new and novel ornamental and utilitarian features of a device for holding a product container such as tissue and glove boxes.

### BACKGROUND OF THE INVENTION

There are many products manufactured today that are generally used once and then discarded, especially in the area of personal hygiene. Good personal hygiene habits, such as washing one's hands, covering one's mouth when one sneezes, drying one's hands with a towel help minimize the spread of bacteria, viruses and associated illnesses. Many disposable product containers have been designed that hold and dispense a large number of individual disposable product pieces such as tissue boxes and rubber glove boxes. Facial tissue (paper handkerchiefs), for example, refers to a class of soft, absorbent, disposable paper that is more suitable for use on the face/nose and come in boxes containing about 100 individual sheets (as one example). Disposable vinyl gloves are also popular with doctors, mechanics, food handling staff, and janitorial workers (to name a few) and come in boxes configured to hold and dispense 100 gloves (or more).

Hand sanitizers are another hygiene product used by people (often in combination with tissue paper). While at least one study has indicated that the use of antibiotic based hand sanitizers resulted in more outbreaks of norovirus-related illnesses, most medical researchers contend that antibiotic hand sanitizers are very useful and an important method used to prevent most bacterial and viral infections, with rare exceptions. Plain liquid soap is known for its hygiene benefits as well. Many such hand sanitizers come in a plastic container that holes about 10 to 20 ounces of product. Such containers are often configured with a pump at the top to dispenser the sanitizer. Notably, the use of hand sanitizers is particularly advantageous when one needs to wipe one's hands with a tissue. Thus, it would be advantageous to store hand sanitizers near the above described tissue paper.

While the sizes of the containers described above may vary, they are generally the same shape and size. In addition, the usefulness of such hygiene products is amplified when such items are disposed in one's environment at locations where they are most likely to be required. One obvious example is near a sink in a bathroom. Consequently, a number prior art devices have been devised to hold product containers in advantages locations such as on walls next to sinks. Such prior art devices, however, do not provide for an apparatus and method to hold a product container in a way that allows for multiple product container orientations. Nor do such prior art containers provide for an apparatus and method for holding both liquid sanitizer products (such as soap and other chemicals) and paper hygiene products.

The inventions disclosed in this document addresses at least such issues.

### SUMMARY OF THE INVENTION

Some of the objects and advantages of the invention will now be set forth in the following description, while other objects and advantages of the invention may be obvious from the description, or may be learned through practice of the invention.

Broadly speaking, a principle object of the present invention is to provide an apparatus and method for supporting a product dispenser at a production distribution point that allows for multiple product dispenser orientations.

Another object of the present invention is to provide an apparatus and method for holding a first product dispenser that allows for multiple product dispenser orientations and that is further configured to hold a second product dispenser.

Additional objects and advantages of the present invention are set forth in the detailed description herein or will be apparent to those skilled in the art upon reviewing the detailed description. Also, it should be further appreciated that modifications and variations to the specifically illustrated, referenced, and discussed steps, or features hereof may be practiced in various uses and embodiments of this invention without departing from the spirit and scope thereof, by virtue of the present reference thereto. Such variations may include, but are not limited to, substitution of equivalent steps, referenced or discussed, and the functional, operational, or positional reversal of various features, steps, parts, or the like. Still further, it is to be understood that different embodiments, as well as different presently preferred embodiments, of this invention may include various combinations or configurations of presently disclosed features or elements, or their equivalents (including combinations of features or parts or configurations thereof not expressly shown in the figures or stated in the detailed description).

Those of ordinary skill in the art will better appreciate the features and aspects of such embodiments, and others, upon review of the remainder of the specification.

### BRIEF DESCRIPTION OF THE DRAWINGS

A full and enabling description of the present subject matter, including the best mode thereof, directed to one of ordinary skill in the art, is set forth in the specification, which makes reference to the appended figures, in which:

FIG. 1 is a side elevated perspective view of one exemplary embodiment thereof;

FIG. 2 is a front elevational view of one exemplary embodiment thereof;

FIG. 3 is a back elevational view of one exemplary embodiment thereof;

FIG. 4 is a first side elevational view of one exemplary embodiment thereof;

FIG. 5 is a second side elevational view of one exemplary embodiment thereof;

FIG. 6 is a top plan view of one exemplary embodiment thereof showing a rectangular access-void;

FIG. 6b is a top plan view of one exemplary embodiment thereof showing a rectangular access-void in a first compartment and a second compartment with no access-void;

FIG. 6c is a top plan view of one exemplary embodiment thereof showing a second compartment with no access-void disposed between two first compartments each showing a rectangular access-void;

FIG. 7 is a bottom plan view of one exemplary embodiment thereof showing a rectangular access-void;

FIG. 8 is a top plan view of one exemplary embodiment thereof showing an elliptical access-void;

FIG. 9 is a bottom plan view of one exemplary embodiment thereof showing a rectangular access-void

FIG. 10a is a side elevational view of the exemplary embodiment depicted in FIG. 6 attached to a support structure defining a distribution point showing the first product dispenser in a first orientation;

FIG. 10b is a side elevational view of the exemplary embodiment depicted in FIG. 6b attached to a support structure defining a distribution point showing the first product dispenser in a second orientation and further showing a second product dispenser disposed in the second compartment;

FIG. 11 is a side elevated perspective view of one exemplary alternative embodiment thereof;

FIG. 12 is a front elevational view of the exemplary embodiment depicted in FIG. 11;

FIG. 13 is a back elevational view of one exemplary embodiment depicted in FIG. 11;

FIG. 14 is a first side elevational view of one exemplary embodiment depicted in FIG. 11;

FIG. 15 is a second side elevational view of one exemplary embodiment depicted in FIG. 11;

FIG. 16 is a top plan view of one exemplary embodiment depicted in FIG. 11 showing a square access-void;

FIG. 17 is a bottom plan view of one exemplary embodiment depicted in 11 showing a square access-void;

FIG. 18 is a top plan view of one exemplary embodiment thereof showing a circular access-void; and

FIG. 19 is a bottom plan view of one exemplary embodiment thereof showing a circular access-void.

### DISCLOSURE OF THE INVENTION

### Detailed Description

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used on another embodiment to yield a still further embodiment. Thus, it is intended that the present invention covers such modifications and variations as come within the scope of the appended claims and their equivalents. Other objects, features, and aspects of the present invention are disclosed in or may be determined from the following detailed description. Repeat use of reference characters is intended to represent same or analogous features, elements or steps. It is to be understood by one of ordinary skill in the art that the present discussion is a description of exemplary embodiments only, and is not intended as limiting the broader aspects of the present invention.

### Construction Aids

For the purposes of this document two or more items are “mechanically associated” by bringing them together or into relationship with each other in any number of ways including a direct or indirect physical “releasable connections” (snaps, screws, Velcro®, bolts, etc.—generally connections designed to be easily and frequently released and reconnected), “hard-connections” (welds, rivets, macular bonds, generally connections that one does not anticipate disconnecting very often if at all and that generally needs to be “broken” to separate), and/or “moveable connections” (rotating, pivoting, oscillating, etc.).

For the purposes of this document, unless otherwise stated, the phrase “at least one of A, B, and C” means there is at least one of A, or at least one of B, or at least one of C or any combination thereof (not one of A, and one of B, and one of C).

This document includes headers that are used for place markers only. Such headers are not meant to affect the construction of this document, do not in any way relate to the meaning of this document nor should such headers be used for such purposes.

While the particulars of the disclosed inventions and associated technology may be described for use with hygiene products, the inventions may be adapted for use with any type of containers for holding product to be dispensed.

### Description

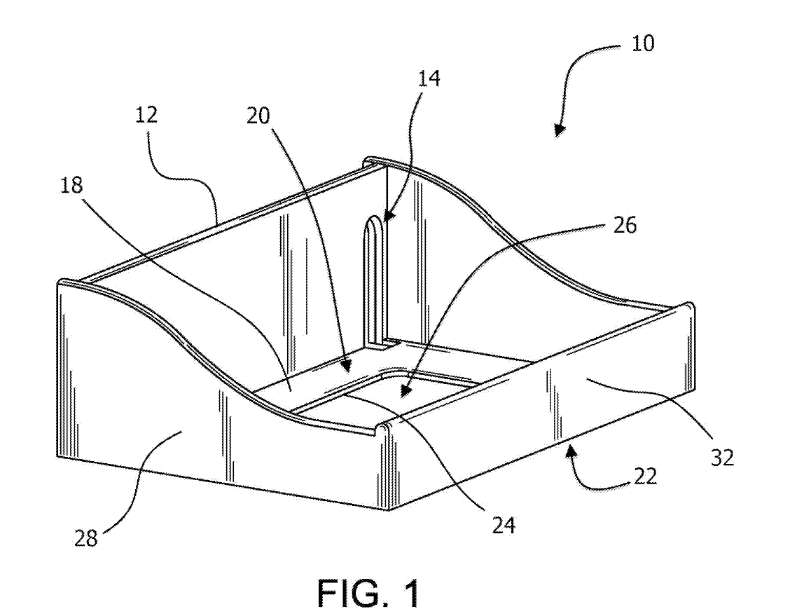

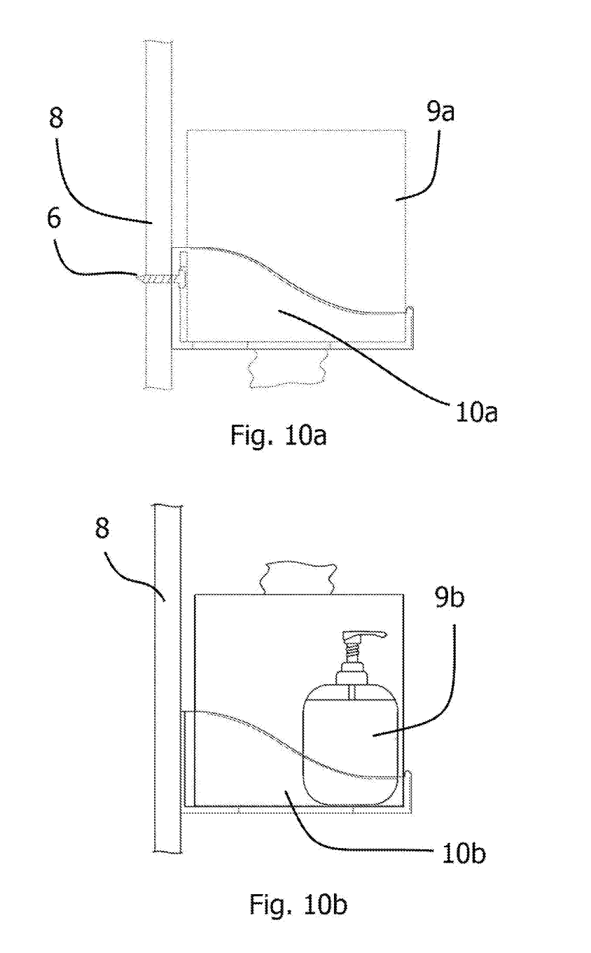

Referring now more particularly to FIG. 1, FIG. 10 and FIG. 11, a product container holder (PCH) configured to support a product container (9, FIG. 10) storing a product to be dispensed is presented. For the currently preferred embodiment, the PCH (10) comprises a first component (12) having a perimeter that defines a polygonal shape. Embodiments of first component (12) include a plates and panels and will be referred to as a back panel. For the embodiment in FIG. 1, such polygonal shape is a rectangle although any polygonal shape (include circles) may be used. The back panel (12) defines at least one attachment interface (14) configured to allow the mechanical association of the back panel to a distribution point. A distribution point is simply any location where a user wishes to store a product to be dispensed. One example would be a point along a wall perhaps above or near a sink. As can be seen in FIG. 1, the attachment interface (14) is a pair simply a recessed grooves suitable for receiving an attachment device (6, FIG. 10). Other types of attachment interfaces may be used such as holes and integral clips. Suitable embodiments of an attachment devices include screws, bolts, nails, clips and any device that can mechanically associate two items together.

PCH (10) further comprises a support component (18). Embodiments of suitable support components include plates and panels and similar items and will be referred to as a support plate (18) defining two opposing surfaces (20, 22) connected by a peripheral edge (24). The outer perimeter of the support plate (18) defines a polygonal shape which is rectangular for the preferred embodiment. One of ordinary skill in the art will appreciate that any polygonal shape (including circles) can be used. The support plate (18) further defines an access-void (26) where the perimeter of the access-void (26) defines a polygonal access-void-shape. The access-vid-shape may or may not be similar to the shape defined by the outer perimeter of the support plate (18). Please note that “similar” polygons have the same shape, but can be different sizes.

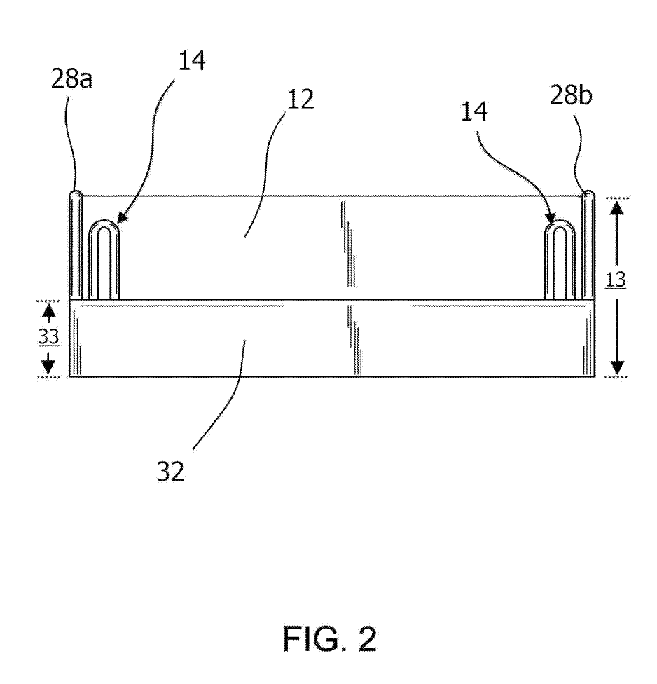

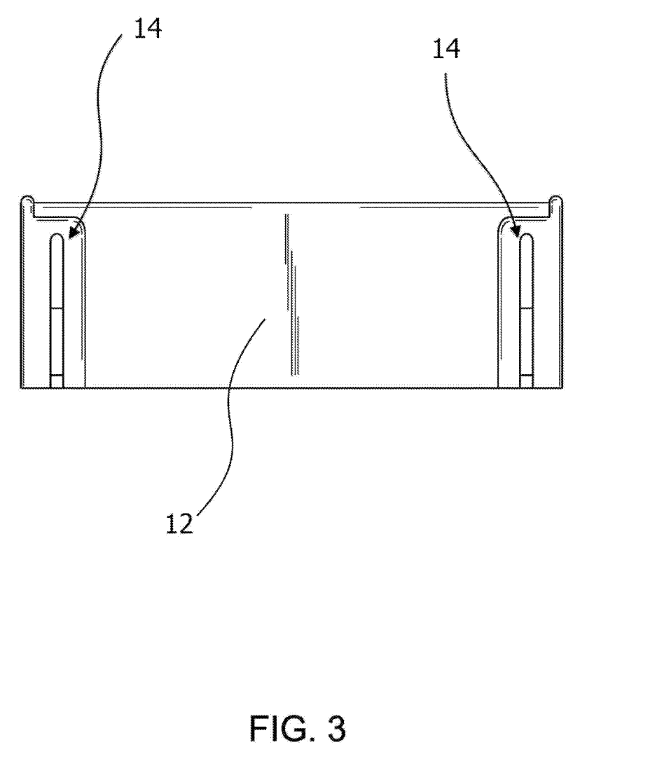

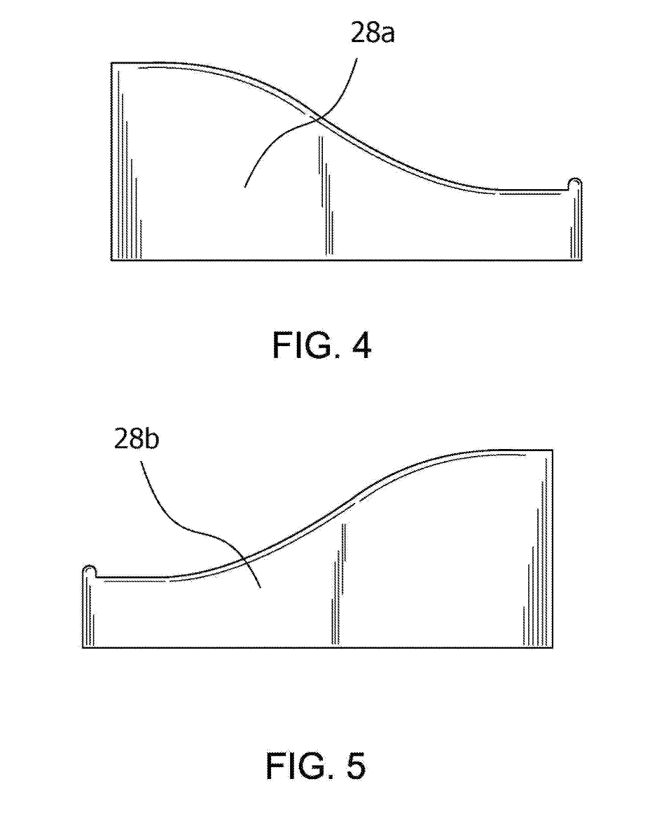

The support plate (18) may be mechanically associated with the back panel (12) or it can be integrally form with the back panel (12). For the disclosed preferred embodiments, the support plate (18) extends perpendicularly away from the back panel (12) so that the height (13) of the back panel is defined by the distance from said support plate (18), at the point the support plate (18) meets the back panel (12), to the opposing edge of said back panel (12) as best seen in FIG. 2 and FIG. 11.

For some embodiments, PCH (10) further comprise a front panel (32) that defines a perimeter having a polygonal shape preferably similar to the polygonal shape defined by the back panel (12). For the embodiments depicted in the various drawings, such front panel polygonal shape defines a rectangular shape. A portion of the front panel (32) is mechanically associated with (or formed integral to) a portion of the support plate (18) so that the front panel extends perpendicularly away from the support plate in the same direction as the back panel (12) as depicted in FIG. 1. The front panel height (33, FIG. 2, FIG. 11) is defined by the distance from said support plate (18), at the point the support plate meets the front panel (32), to the opposing edge of said front panel (32). While the relative heights of the back panel and front panel are mostly determined by ornamental tastes, for the embodiments depicted in the drawings, the front panel height (33) is about ⅓ the back-panel height (13).

For some embodiments, PCH (10) further comprise side panels (collectively referred to as side panels (28)). As depicted in FIG. 1 and FIG. 2, a first side panel (28a) and second side panel (28b) are each mechanically associated with (or formed integral to) the back panel (12), the support plate (18) and the front panel (32) to define an open box structure where the support plate (18) defines the bottom of such open box structure. As noted above, for one embodiment, the back-panel height (13) is three times longer than the front panel height (33). For such embodiments, the top edge of the first and second side panels (28) each extend from the top of the back-panel down to the top of said front panel to define a side panel slope shape. While the type of slope shape used is mostly determined by ornamental tastes, for the currently preferred disclosed embodiments the side panel slope shape is as shown in the various figures and is called a shallow S-Shape.

As noted previously, a “distribution point” is simply any location where a user wishes to store a product to be dispensed. As best seen in FIG. 10, for the above PCH (10) configuration, the attachment interface (14) is configured for being mechanically associated with a distribution point (e.g. wall) so that the support plate (18) is oriented to provide support for a product dispenser (such as a tissue box or glove box) above access-void (26). One example is a tissue box that defines a box-void where a section of a tissue paper extends partly out of the box-void to allow a user to pull that tissue paper out of the box when needed. Such box-void is generally defined by what is normally considered the top of the tissue box. The present invention, however, allows such “top” to be either the top or bottom as explained below.

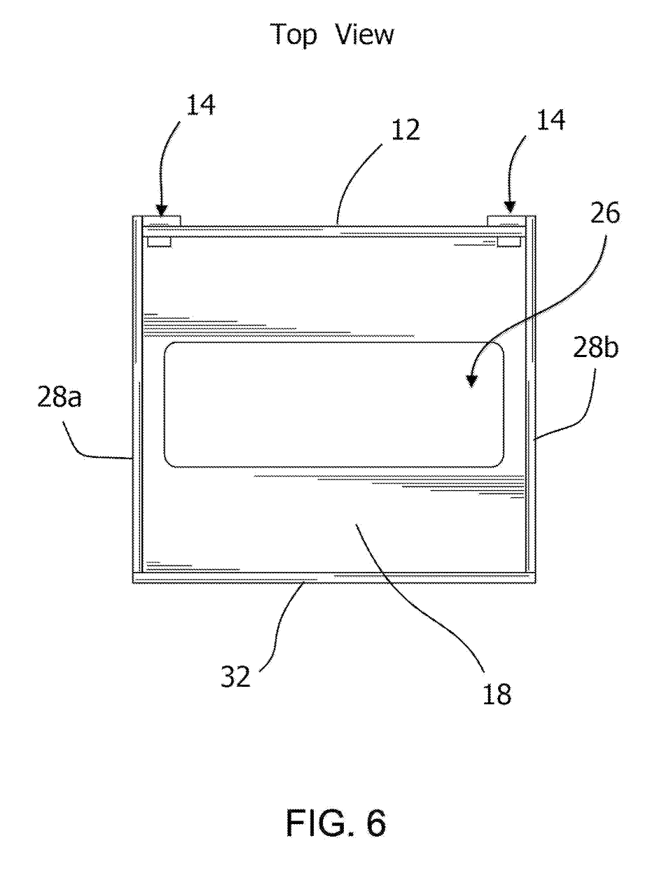

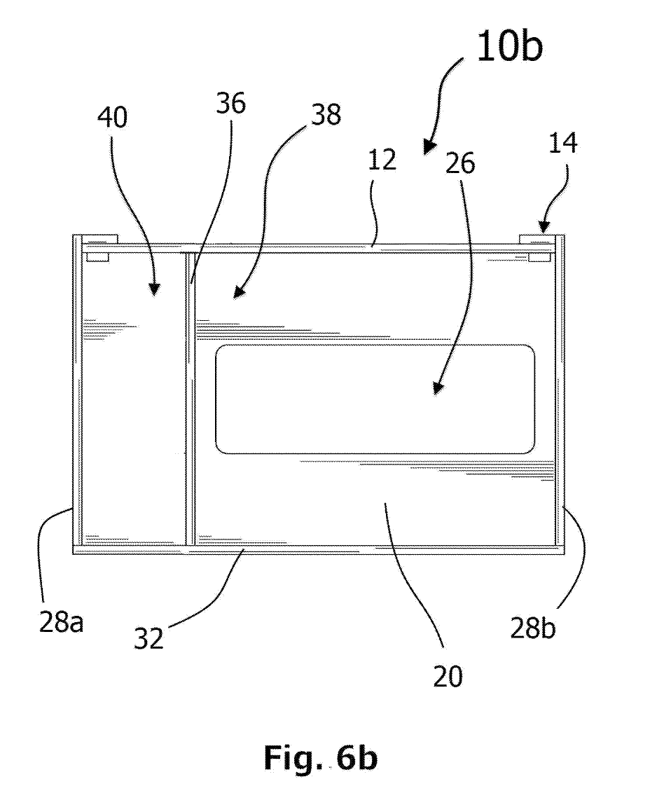

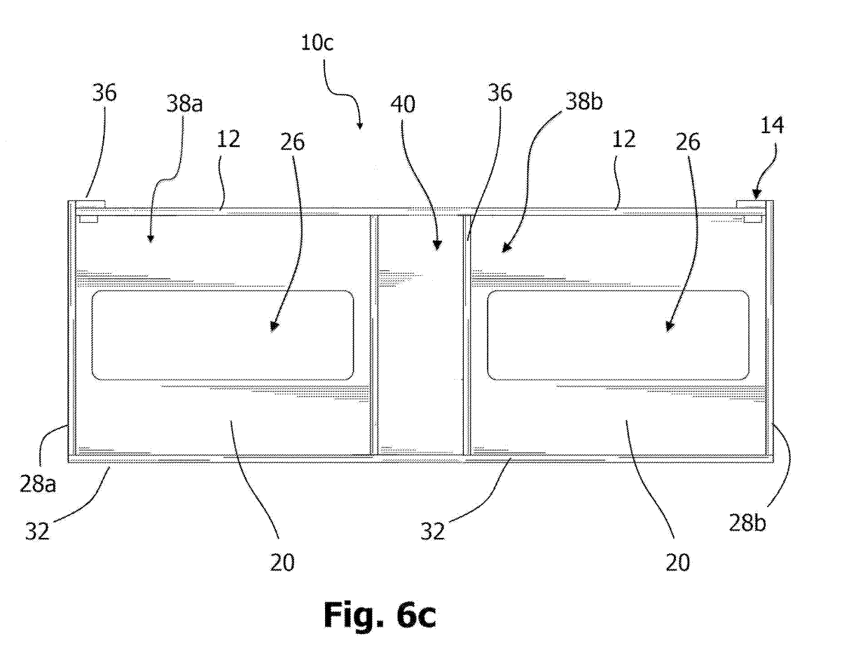

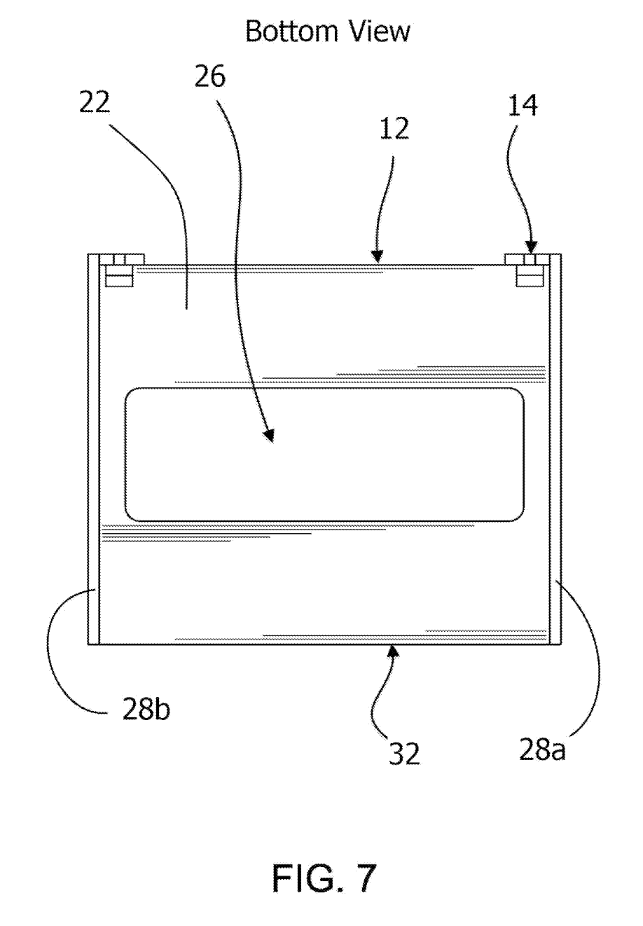

As best seen in FIG. 6 and FIG. 7, the access-void (26) is suitably sized and disposed along the support plate (18) to allow for two product dispenser orientations. As best seen in FIG. 10b, for a first product dispenser orientation the product dispenser (9a) can be disposed/placed on the support plate (18) to allow the bottom (with no hole) of the product dispenser (9a) to sit adjacent to the access-void (26) so that the product is removed from the “top” of the product dispenser. As best seen in FIG. 10b, for a second product dispenser orientation, the access-void (26) is adjacent to a dispenser-box-void defined by the product dispenser so that a product stored inside the product dispenser can exit said dispenser-box-void and through the access-void (26). Basically, the current invention allows what is normally the “top” of a product dispenser to become the “bottom” and vice versa.

### One Integral Piece

It will be appreciated that the various components of the PCH (10) device may be formed of one integral piece without departing from the scope and spirit of the invention. For such configurations, the various PHC (10) features are preferably formed using an injection moulding process or a 3-D printing processing. The well-known injection moulding process is a manufacturing process for producing parts by injecting material into a mould. Injection moulding can be performed with a host of materials mainly including metals, (for which the process is called die-casting), glasses, elastomers, confections, and most commonly thermoplastic and thermosetting polymers. For the preferred embodiment, the material used is plastic.

### Multi-Compartment PCH

One alternative embodiment of the invention is presented in FIGS. 6b and 10b which may be referred to as a hygiene station structure configured of holding a plurality of product dispensers. For this embodiment, the PCH (10b) device further comprises a partition-panel (36) preferably having the same general shape of the side panels (28). The partition-panel (36) is disposed between and in alignment with the side panels (28) and mechanically associated with the back-panel (12) and said front panel (32) to define a first compartment (38) and a second compartment (40). Basically, the second compartment has been added to the PCH (10) device. As depicted in FIG. 6b, for one preferred embodiment the access-void (26) is positioned in the center of the first compartment (38) and does not extend into the second compartment (40). That said, embodiments where the second compartment (40) defines a separate void or where the access-void (26) extends into the second compartment fall within the scope and spirit of the invention.

As depicted in FIG. 10b, second compartment (40) may be particularly useful for holding a second type of hygiene product (9b) such as liquid soap or an antibiotic gel. Thus, the two compartments may be of different sizes. For the embodiment depicted in FIG. 6b, the first compartment (38) is about four times wider (distance from the side panel to the partition-panel (36) than the second compartment and where the distance from the front panel (32) and the back-panel (12) are equal.

For yet another embodiment, not shown, a second partition panel may be included so that third compartment is defined of the same size as the second compartment and disposed on the opposite side of the first compartment to define a symmetrical structure with two side compartments and a center compartment. For such embodiment, the PCH (10) comprises two “second” compartments (40) separated by a “first compartment” (38) disposed in the center. For such configuration, the PCH further comprising a second partition-panel having the same general shape of said first partition panel (36) where such second partition-panel (36) is disposed between the access-void (26) and in alignment with the side panels (28) and mechanically associated with (or formed integral to) said back-panel (12) and front panel (32) to define the “third” compartment of equal size to the second compartment wherein said first compartment is defined between the second and third compartment.

For yet another embodiment, PCH (10c, FIG. 6c) comprises two “first” compartments (38) with a “second compartment” (40) disposed in the center. For such configuration, PCH (10c) further comprising a second partition-panel (36) having the same general shape of the first partition panel (36) where such second partition-panel (36) is disposed between the access-void (26) and a side panel and in alignment with the side panels (28). The second partition-panel (36) is mechanically associated with said back-panel (12) and front panel (32) to define a third compartment of equal size to the first compartment (38) wherein said second compartment (40) is defined between the two first compartments (38).

### Access-Void Shapes

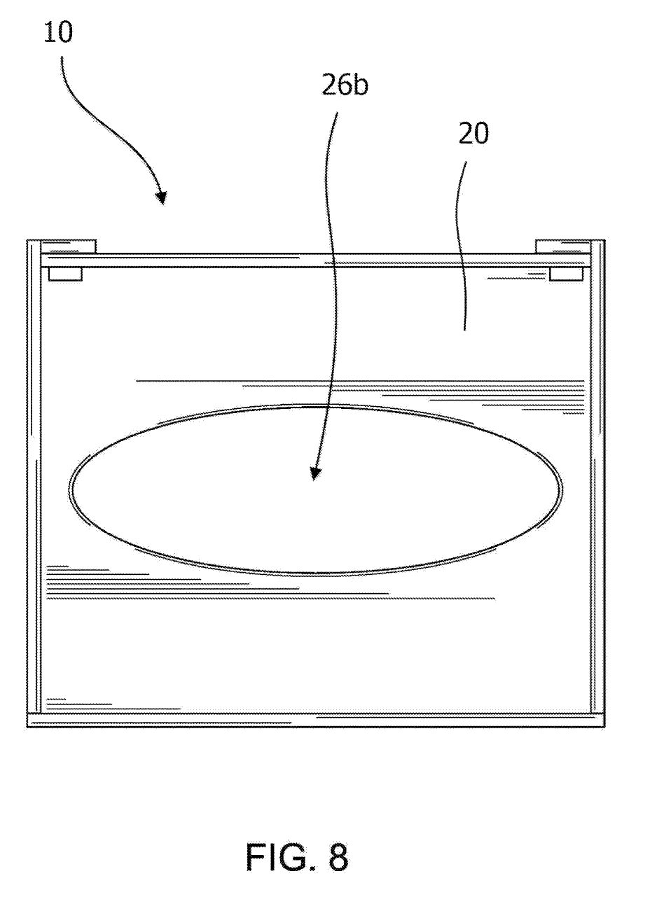

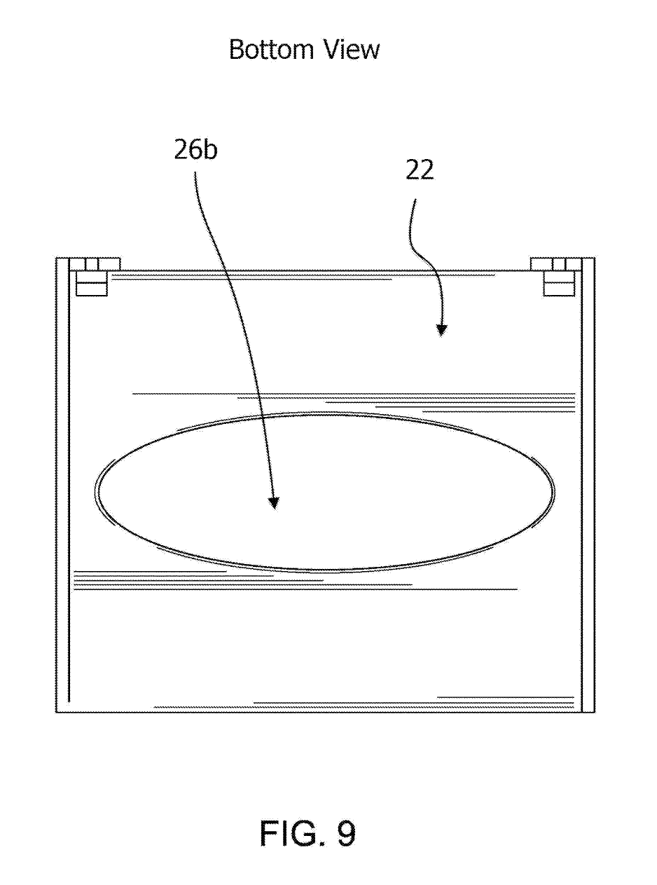

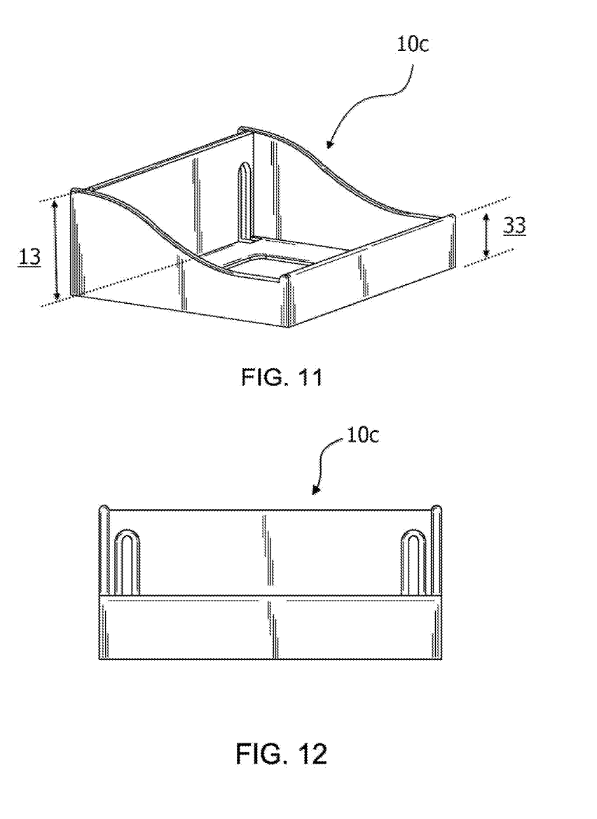

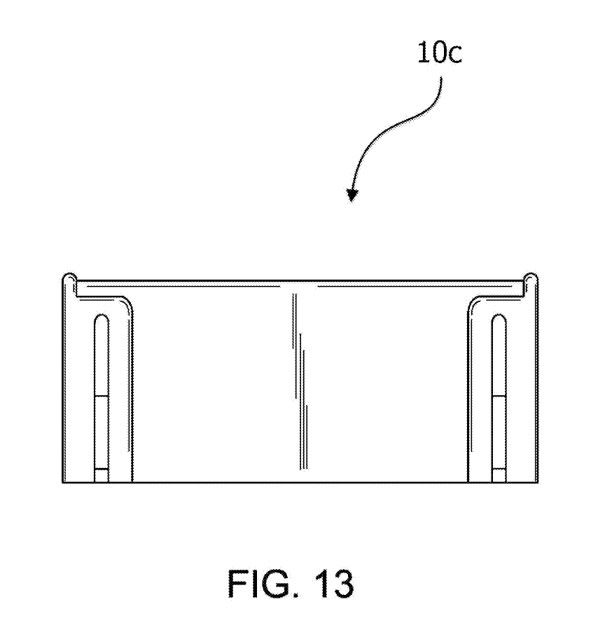

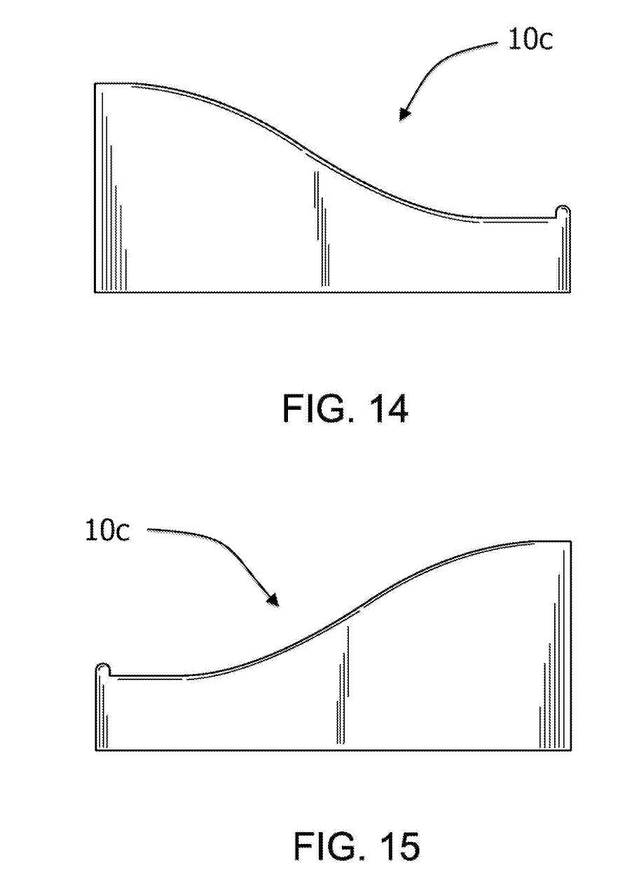

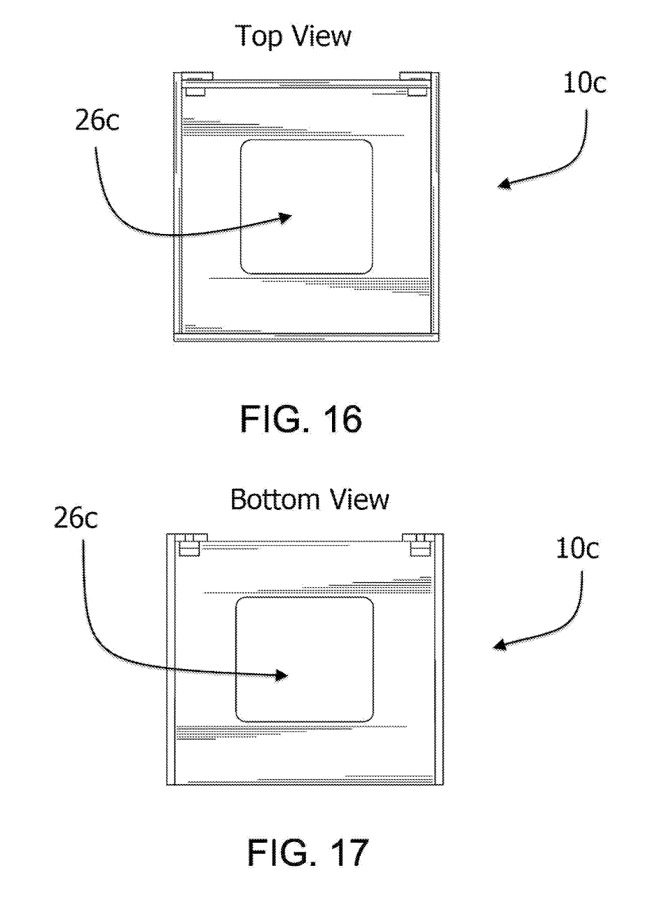

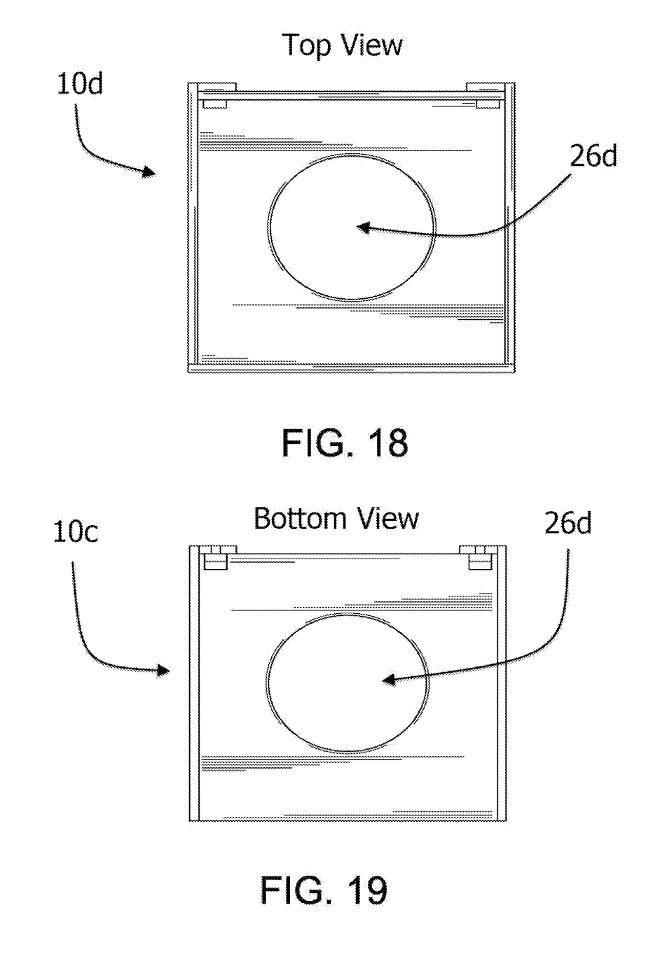

The perimeter of access-void (26) may define any shape desired and may be determined by either ornamental tastes. As depicted in FIG. 8, and FIG. 9, the perimeter of access-void (26) may define an ellipsis or prolate spheroid type shape (which are considered polygons for this document as all enclosed line shapes are formed with a plurality of straight lines if one zooms in close enough). Similarly, FIG. 11-FIG. 17 present a PCH (10) device with a square access-void (26) while FIGS. 18 and 19 show a round access-void (26).

### Ornamental Features

As noted above, many of the various shapes and panels and voids defined by the PCH (10) device are determined more by aesthetics than by utilitarian needs. For example, the difference in height of the back-panel (12) and the front panel (32) is determined more by aesthetics than utilitarian needs. Additionally, while the side panels are constructed to slope from the top of the back panel down to the top of the front panel such is not a utilitarian requirement. For example, the outer perimeters of the side panels could define a rectangle that is taller than the height of the front panel where the front panel and the side panels meet. Similarly, for the preferred embodiment the side panels extend from the top of the back-panel (12) to the top of the front panel (32) to define a shallow S-Shape. Such is not the utilitarian requirement that one of aesthetics. The PCH (10) devices may further be constructed of translucent/transparent material which may or may not define a color. Additionally, surface patterns may be applied to the outer surface of the PCH (10) device to achieve a desired look/feel.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention as claimed.

What is claimed is:

1. A product container holder configured to support a container storing a product to be dispensed, said product container holder comprising:
a first component having a perimeter that defines a polygonal shape, said first component defining an attachment interface configured for mechanically associating said first component to a distribution point;
a support component defining two opposing surfaces defining a support plate and wherein the outer perimeter of said support plate defines a polygonal shape and wherein said support plate further defines a polygonal shaped access-void therethrough and wherein said support component is one of (a) mechanically associated with and (b) integral to said first component so that said support plate extends perpendicularly away from said first component;
wherein said attachment interface is configured for being mechanically associated with said distribution point so that said support plate is oriented to provide support for a product container above said access-void; and
wherein said access-void is suitably sized and disposed along said support plate so that one of (a) said product container can be disposed on said support plate in a first orientation that allows the product to be dispensed to extend out of said product container and away from said access-void and (b) said product container can be disposed on said support surface in a second orientation so that said access-void is adjacent to a container-void defined by the product container so that the product to be dispensed extends out of said container-void and through said access-void.

2. A product container holder as in claim 1, further comprising a front panel having a peripheral edge that defines a rectangular shape wherein a portion of the front panel is associated with a portion of the support component peripheral edge and opposed to said first component so that said front plate extends perpendicularly away from said support plate in the same direction as said first component.

3. A product container holder as in claim 2, further comprising a first and second side panel wherein each side panel is associated with said first component, said support component and said front panel to define an open box structure wherein said support component defines the bottom of said open box structure.

4. A product container holder as in claim 3, wherein said first component defines a back-panel having a rectangular outer perimeter wherein the height of said back-panel is longer than the height of said front panel and wherein the first and second side panels extend from the top of said back-panel down to the top of said front panel to define a side panel slope shape.

5. A product container holder as in claim 6, wherein the height of said back-panel is 3 times longer than the height of said front panel and wherein the side panels slope shape defines an shallow S-Shape.

6. A product container holder as in claim 3, further comprising a partition-panel having the same general shape of said first panel and said second panel wherein said partition-panel is disposed between and in alignment with said first panel and said second panel and associated with said side bank-panel and said front panel to define a first compartment and a second compartment.

7. A product container holder as in claim 6, wherein said access-void is positioned in the center of said first compartment and does not extend into said second compartment.

8. A product container holder as in claim 6, wherein said first compartment is 4 times wider than said second compartment.

9. A hygiene station structure configured to support and hold hygiene product dispensers, said hygiene station structure comprising:
a back panel having a perimeter that defines a polygonal shape, said back panel defining at least one attachment interface configured to mechanically associate said back panel to a distribution point;
a support plate defining two opposing surfaces wherein the outer perimeter of said support plate defines a polygonal shape and wherein said support plate further defines an access-void therethrough defining a polygonal access-void-shape and wherein said support plate is one of (a) mechanically associated with said back panel and (b) integral to said back panel so that said support plate extends perpendicularly away from said back panel and wherein the height of said back panel is defined by the distance from said support plate, at the point the support plate meets the back panel, to the opposing edge of said back panel;
a front panel with a perimeter that defines a polygonal shape similar to the polygonal shape defined by said back panel, wherein a portion of the front panel is associated with a portion of the support plate so that said front panel extends perpendicularly away from said support plate in the same direction as said back panel and wherein the height of said front panel is defined by the distance from said support plate, at the point the support plate meets the front panel, to the opposing edge of said front panel;
a first and second side panel wherein each side panel is associated with said back panel, said support plate and said front panel to define an open box structure wherein said support plate defines the bottom of said open box structure;
wherein said attachment interface is configured for being mechanically associated with said distribution point so that said support plate is oriented to provide support for a hygiene product dispenser above said access-void; and
wherein said access-void is suitably sized and disposed along said support plate so that one of (a) said hygiene product dispenser can be disposed on said support plate in a first orientation that allows the bottom of the hygiene product dispenser to sit adjacent to said access-void, and (b) said hygiene product dispenser can be disposed on said support surface in a second orientation so that said access-void is adjacent to a dispenser-void defined by the hygiene product dispenser so that a product stored inside said hygiene product dispenser can exit said dispenser-void and through said access-void.

10. A hygiene station structure as in claim 9, wherein said back-panel defines a rectangular perimeter and wherein the front panel defines a rectangular perimeter and wherein the height of said back-panel is longer than the height of said front panel.

11. A hygiene station structure as in claim 10, wherein the first and second side panels each extend from the top of said back panel down to the top of said front panel to define a side panel slope shape.

12. A hygiene station structure as in claim 11, wherein the height of said back-panel is 3 times longer than the height of said front panel and wherein the side panels slope shape defines a shallow S-Shape.

13. A hygiene station structure as in claim 11, further comprising a partition-panel having the same general shape of said first side panel and said second side panel wherein said partition-panel is disposed between and in alignment with said first side panel and said second side panel and associated with said back-panel and said front panel to define a first compartment and a second compartment.

14. A hygiene station structure as in claim 13, wherein said access-void is positioned in the center of said first compartment and does not extend into said second compartment.

15. A hygiene station structure as in claim 14, wherein said first compartment is 4 times wider than said second compartment.

16. A hygiene station structure as in claim 15, further comprising a second partition-panel having the same general shape of said first partition panel and wherein said second partition-panel is disposed between said access-void and in alignment with said first side panel and said second side panel and associated with said back-panel and said front panel to define a third compartment of equal size to said second compartment wherein said first compartment is defined between said second and third compartment.

17. A one-piece product container holder configured to support a product-container storing a product to be dispensed, said one piece product container holder comprising:
a back-panel having a perimeter that defines a polygonal shape, said back-panel defining an attachment interface configured for mechanically associating said back-panel to a distribution point;
a support plate defining two opposing surfaces wherein the perimeter of said support plate defines a polygonal shape and wherein said support plate further defines a polygonal shaped access-void therethrough and wherein said support-plate is associated said back-panel so that said support plate extends perpendicularly away from said back-panel;
a front panel having perimeter that defines a rectangular shape wherein a portion of the front panel is associated with a portion of the support plate that is opposed to said back-panel so that said front plate extends perpendicularly away from said support plate in the same direction as said back-panel;
wherein said attachment interface disposed along said back-panel so that the back-panel is mechanically associated with said distribution point so that said support plate is oriented to provide support for a product container above said access-void; and
wherein said access-void is suitably sized and disposed along said support plate so that one of (a) said product container can be disposed on said support plate in a first orientation that allows the product to be dispensed to extend out of said product container and away from said access-void and (b) said product container can be disposed on said support surface in a second orientation so that said access-void is adjacent to a container-void defined by the product container so that the product to be dispensed extends out of said container-void and through said access-void.

18. A one-piece product container holder as in claim 17, further comprising a first side panel and second side panel wherein each side panel is associated with said back-panel, said support plate and said front panel to define an open box structure wherein said support plate defines the bottom of said open box structure.

19. A one-piece product container holder as in claim 3, wherein said back-panel defines a height that is longer than the height of said front panel and wherein the first and second side panels extend from the top of said back-panel down to the top of said front panel to define a side panel slope shape.

20. A one-piece product container holder as in claim 19, further comprising a partition-panel having the same general shape of said first side panel and said side second panel wherein said partition-panel is disposed between and in alignment with said first side panel and said second side panel and associated with said side bank-panel and said front panel to define a first compartment and a second compartment and wherein said access-void is positioned in the center of said first compartment and does not extend into said second compartment.
